(19) United States
(12) Patent Application Publication
(10) Pub. No.: US 2022/0346490 A1
(43) Pub. Date: Nov. 3, 2022

(54) Enhancing Exercise Through Augmented Reality

(71) Applicant: NIKE, Inc., Beaverton, OR (US)

(72) Inventor: Craig Rowley, Beaverton, OR (US)

(21) Appl. No.: 17/848,940

(22) Filed: Jun. 24, 2022

Related U.S. Application Data
(63) Continuation of application No. 15/165,881, filed on May 26, 2016, now abandoned.
(60) Provisional application No. 62/168,308, filed on May 29, 2015.

(51) Int. Cl. A43B 3/34; G06Q 50/00; A61B 5/00; A61B 5/11; G06Q 50/20; G06Q 30/02; G16H 20/30

(52) U.S. Cl. CPC A43B 3/34; G06Q 50/01; A61B 5/744; A61B 5/1123; G06Q 50/20; G06Q 30/0282; G16H 20/30

(57) ABSTRACT

The disclosure relates to enhancing exercise through augmented reality. In particular, the disclosure describes monitoring a user's performance and generating a virtual representation of that user's performance to be displayed during a future exercise routine to motivate the user to improve performance during their next workout.

This application is a continuation of U.S. patent application Ser. No. 15/165,881 filed May 26, 2016, which claims priority to provisional U.S. Application No. 62/168,308 filed May 29, 2015, the disclosure and contents of which are hereby incorporated by reference in their entirety.

### BACKGROUND

While most people appreciate the importance of physical fitness, many have difficulty finding the motivation required to maintain a regular exercise program or to continually improve their workouts during their exercise routines. In addition, some people find it particularly difficult to maintain an exercise regimen that involves continuously repetitive motions, such as running, walking and bicycling.

Moreover, individuals may view exercise as work or a chore and thus, separate it from enjoyable aspects of their daily lives. Often, this separation between athletic activity and other activities reduces the amount of motivation that an individual might have toward exercising. Further, athletic activity services and systems directed toward encouraging individuals to engage in athletic activities might also be too focused on one or more particular activities while an individual's interests are ignored. This may further decrease a user's interest in participating in athletic activities or using the athletic activity services and systems.

Therefore, improved systems and methods to address these and other shortcomings in the art are desired.

### BRIEF SUMMARY

The following presents a simplified summary in order to provide a basic understanding of some aspects of the disclosure. The summary is not an extensive overview of the disclosure. It is neither intended to identify key or critical elements of the disclosure nor to delineate the scope of the disclosure. The following summary merely presents some concepts of the disclosure in a simplified form as a prelude to the description below.

Aspects of this disclosure relate to motivating individuals to maintain or improve upon a threshold level of physical activity. Certain implementations may motivate individuals by informing users of their current progress against user defined and system defined goals. In one embodiment, feedback may facilitate individuals observing one or more benefits associated with physical activity. By realizing benefits associated with their activities, users may be encouraged to continue exercising or increase exercising intensity.

In an embodiment, a visual and audio system may motivate users to push their limits by extending workouts by a known quantifiable amount. The system may engage users by enabling them to explore new variations of their common workouts while eliminating worry or fear of the unknown by providing a trusted system to recommend safe modifications to their workout routines or exercise programs.

Example embodiments may relate to a system, method, apparatus, and computer readable media configured for monitoring a user's performance during an exercise routine. In an embodiment, the monitored performance may be used to generate a virtual representation of the user's performance to be displayed during a future exercise routine to motivate the user to improve performance during their next workout. In another embodiment, a virtual shadow may illustrate a proper form (or any specific form) of the exercise to assist the user with improving performance during their workout routine.

In an embodiment, an electronic device capable of communicating with a user may overlay information into a user's field of vision through use of eyewear or other personal wearable items. Such received information may include audio information received from speakers or other sound producing devices. In an embodiment, the overlay may include a virtual representation of a user's prior workout performance. In an embodiment, a user may compete against their prior workout performances or against a friends or athletes prior workout performance. In yet another embodiment, multiple workout performances may be represented by different avatars or visual representations displayed in a user's field of vision as the user completes his/her current workout.

These and other aspects of the embodiments are discussed in greater detail throughout this disclosure, including the accompanying drawings.

### BRIEF DESCRIPTION OF THE DRAWINGS

The present disclosure is illustrated by way of example and not limited in the accompanying figures in which like reference numerals indicate similar elements.

FIGS. 1A-B illustrate an exemplary system for providing an enhanced workout for a user in accordance with example embodiments, wherein FIG. 1A illustrates an example network configured to monitor and provide feedback to a user performing various athletic activities, and FIG. 1B illustrates an example computing device in accordance with example embodiments of the disclosure.

FIGS. 2A, 2B, and 2C illustrate example sensory and feedback devices that may be worn by a user in accordance with example embodiments of the disclosure.

FIG. 3 illustrates a virtual representation of a user's performance in accordance with example embodiments of the disclosure.

FIG. 4 illustrates example points on a user's body to monitor and provide feedback in accordance with example embodiments of the disclosure.

FIG. 5 illustrates a device providing information to a user during a workout in accordance with example embodiments of the disclosure.

FIG. 6 illustrates a method of generating and displaying avatars in accordance with example embodiments of the disclosure.

FIGS. 7-11 illustrate another exemplary operating environment which may be used with various aspects of the disclosure.

### DETAILED DESCRIPTION

In the following description of the various embodiments, reference is made to the accompanying drawings, which form a part hereof, and in which is shown by way of illustration various embodiments in which the disclosure may be practiced. It is to be understood that other embodiments may be utilized and structural and functional modifications may be made without departing from the scope and spirit of the present disclosure. Further, headings within this disclosure should not be considered as limiting aspects of the disclosure. Those skilled in the art with the benefit of this disclosure will appreciate that the example embodiments are not limited to the example headings.

In an aspect of the disclosure, a user's performance is monitored and a virtual representation of that user's performance is generated to be displayed during a future exercise routine to motivate the user to improve performance during their next workout. In another embodiment, a virtual shadow may illustrate a proper form (or any specific form) of the exercise in real-time feedback to assist the user with improving performance during their workout routine. In an embodiment, an electronic device capable of communicating with a user may overlay information into a user's field of vision through use of eyewear or other personal wearable items during exercise. In an embodiment, a user may compete against their prior workout performances or against a friends or athletes prior workout performance. In yet another embodiment, multiple workout performances may be represented by different avatars or visual representations displayed in a user's field of vision as the user completes his/her current workout.

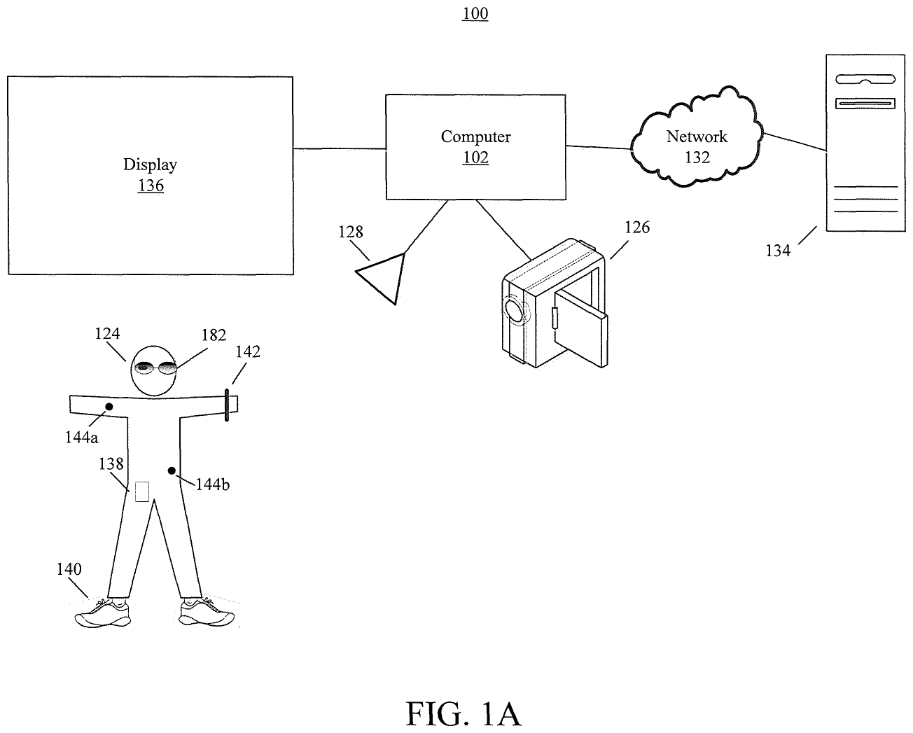

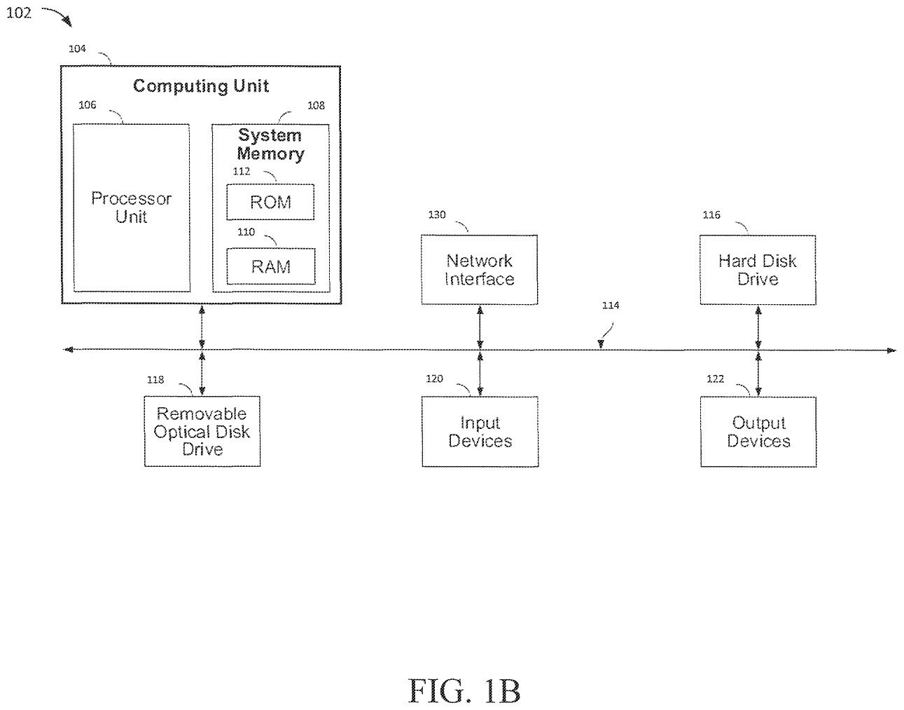

FIG. 1A illustrates an example of a monitoring and feedback system 100 in accordance with example embodiments. Example system 100 may include one or more electronic devices, such as computer 102. Computer 102 may comprise a mobile terminal, such as a telephone, music player, tablet, netbook or any portable device. In other embodiments, computer 102 may comprise a set-top box (STB), desktop computer, digital video recorder(s) (DVR), computer server(s), and/or any other desired computing device. In certain configurations, computer 102 may comprise a gaming console, such as for example, a Microsoft® XBOX, Sony® Playstation, and/or a Nintendo® Wii gaming consoles. Those skilled in the art will appreciate that these are merely example consoles for descriptive purposes and this disclosure is not limited to any console or device.

Turning briefly to FIG. 1B, computer 102 may include computing unit 104, which may comprise at least one processing unit 106. Processing unit 106 may be any type of processing device for executing software instructions, such as for example, a microprocessor device. Computer 102 may include a variety of non-transitory computer readable media, such as memory 108. Memory 108 may include, but is not limited to, random access memory (RAM) such as RAM 110, and/or read only memory (ROM), such as ROM 112. Memory 108 may include any of: electronically erasable programmable read only memory (EEPROM), flash memory or other memory technology, CD-ROM, digital versatile disks (DVD) or other optical disk storage, magnetic storage devices, or any other medium that can be used to store the desired information and that can be accessed by computer 102.

The processing unit 106 and the system memory 108 may be connected, either directly or indirectly, through a bus 114 or alternate communication structure to one or more peripheral devices. For example, the processing unit 106 or the system memory 108 may be directly or indirectly connected to additional memory storage, such as a hard disk drive 116, a removable magnetic disk drive, an optical disk drive 118, and a flash memory card, as well as to input devices 120, and output devices 122. The processing unit 106 and the system memory 108 also may be directly or indirectly connected to one or more input devices 120 and one or more output devices 122. The output devices 122 may include, for example, a monitor display, television, printer, stereo, or speakers. The input devices 120 may include, for example, a keyboard, touch screen, a remote control pad, a pointing device (such as a mouse, touchpad, stylus, trackball, or joystick), a scanner, a camera or a microphone. In this regard, input devices 120 may comprise one or more sensors configured to sense, detect, and/or measure athletic movement from a user, such as user 124, shown in FIG. 1A.

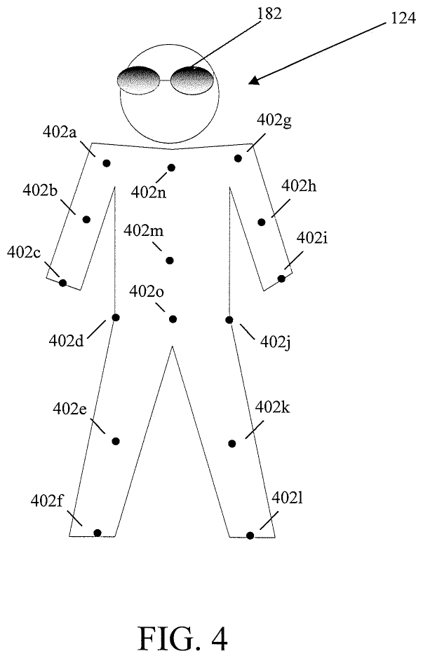

Looking again to FIG. 1A, image-capturing device 126 and/or sensor 128 may be utilized in detecting and/or measuring athletic movements of user 124. In one embodiment, data obtained image-capturing device 126 or sensor 128 may directly detect athletic movements, such that the data obtained from image-capturing device 126 or sensor 128 is directly correlated to a motion parameter. For example, and with reference to FIG. 4, image data from image-capturing device 126 may detect that the distance between sensor locations 402g and 402i has decreased and therefore, image-capturing device 126 alone may be configured to detect that user's 124 right arm has moved. Yet, in other embodiments, data from image-capturing device 126 and/or sensor 128 may be utilized in combination, either with each other or with other sensors to detect and/or measure movements. Thus, certain measurements may be determined from combining data obtained from two or more devices. Image-capturing device 126 and/or sensor 128 may include or be operatively connected to one or more sensors, including but not limited to: an accelerometer, a gyroscope, a location-determining device (e.g., GPS), light sensor, temperature sensor (including ambient temperature and/or body temperature), heart rate monitor, image-capturing sensor, moisture sensor and/or combinations thereof. Example uses of illustrative sensors 126, 128 are provided. Computer 102 may also use touch screens or image capturing device to determine where a user is pointing to make selections from a graphical user interface. One or more embodiments may utilize one or more wired and/or wireless technologies, alone or in combination, wherein examples of wireless technologies include Bluetooth® technologies, Bluetooth® low energy technologies, and/or ANT technologies.

Still further, computer 102, computing unit 104, and/or any other electronic devices may be directly or indirectly connected to one or more network interfaces, such as example interface 130 (shown in FIG. 1B) for communicating with a network, such as network 132. In the example of FIG. 1B, network interface 130, may comprise a network adapter or network interface card (NIC) configured to translate data and control signals from the computing unit 104 into network messages according to one or more communication protocols, such as the Transmission Control Protocol (TCP), the Internet Protocol (IP), and the User Datagram Protocol (UDP). These protocols are well known in the art, and thus will not be discussed here in more detail. An interface 130 may employ any suitable connection agent for connecting to a network, including, for example, a wireless transceiver, a power line adapter, a modem, or an Ethernet connection. Network 132, however, may be any one or more information distribution network(s), of any type(s) or topography(s), alone or in combination(s), such as internet(s), intranet(s), cloud(s), LAN(s). Network 132 may be any one or more of cable, fiber, satellite, telephone, cellular, wireless, etc. Networks are well known in the art, and thus will not be discussed here in more detail. Network 132 may be variously configured such as having one or more wired or wireless communication channels to connect one or more locations (e.g., schools, businesses, homes, consumer dwellings, network resources, etc.), to one or more remote servers 134, or to other computers, such as similar or identical to computer 102. Indeed, system 100 may include more than one instance of each component (e.g., more than one computer 102, more than one display 136, etc.).

Regardless of whether computer 102 or other electronic device within network 132 is portable or at a fixed location, it should be appreciated that, in addition to the input, output and storage peripheral devices specifically listed above, the computing device may be connected, such as either directly, or through network 132 to a variety of other peripheral devices, including some that may perform input, output and storage functions, or some combination thereof. In certain embodiments, a single device may integrate one or more components shown in FIG. 1A. For example, a single device may include computer 102, image-capturing device 126, sensor 128, display 136 and/or additional components. In one embodiment, sensor device 138 may comprise a mobile terminal having a display 136, image-capturing device 126, and one or more sensors 128. Yet, in another embodiment, image-capturing device 126, and/or sensor 128 may be peripherals configured to be operatively connected to a media device, including for example, a gaming or media system. Thus, it goes from the foregoing that this disclosure is not limited to stationary systems and methods. Rather, certain embodiments may be carried out by a user 124 in almost any location.

Computer 102 and/or other devices may comprise one or more sensors 126, 128 configured to detect and/or monitor at least one fitness parameter of a user 124. Sensors 126 and/or 128 may include but not limited to: an accelerometer, a gyroscope, a location-determining device (e.g., GPS), light sensor, temperature sensor (including ambient temperature and/or body temperature), heart rate monitor, image-capturing sensor, moisture sensor and/or combinations thereof. Network 132 and/or computer 102 may be in communication with one or more electronic devices of system 100, including for example, display 136, an image capturing device 126 (e.g., one or more video cameras), and sensor 128, which may be an infrared (IR) device. In one embodiment sensor 128 may comprise an IR transceiver. For example, sensors 126, and/or 128 may transmit waveforms into the environment, including towards the direction of user 124 and receive a “reflection” or otherwise detect alterations of those released waveforms. In yet another embodiment, image-capturing device 126 and/or sensor 128 may be configured to transmit and/or receive other wireless signals, such as radar, sonar, and/or audible information. Those skilled in the art will readily appreciate that signals corresponding to a multitude of different data spectrums may be utilized in accordance with various embodiments. In this regard, sensors 126 and/or 128 may detect waveforms emitted from external sources (e.g., not system 100). For example, sensors 126 and/or 128 may detect heat being emitted from user 124 and/or the surrounding environment. Thus, image-capturing device 126 and/or sensor 128 may comprise one or more thermal imaging devices. In one embodiment, image-capturing device 126 and/or sensor 128 may comprise an IR device configured to perform range phenomenology. As a non-limited example, image-capturing devices configured to perform range phenomenology are commercially available from Flir Systems, Inc. of Portland, Oreg. Although image capturing device 126 and sensor 128 and display 136 are shown in direct (wirelessly or wired) communication with computer 102, those skilled in the art will appreciate that any may directly communicate (wirelessly or wired) with network 132.

User 124 may possess, carry, and/or wear any number of electronic devices, including sensory devices 138, 140, 142, 144 and/or 182. In certain embodiments, one or more devices 138, 140, 142, 144, 182 may not be specially manufactured for fitness or athletic purposes. Indeed, aspects of this disclosure relate to utilizing data from a plurality of devices, some of which are not fitness devices, to collect, detect, and/or measure athletic data. In one embodiment, device 138 may comprise a portable electronic device, such as a telephone or digital music player, including an IPOD®, IPAD®, or iPhone®, brand devices available from Apple, Inc. of Cupertino, Calif. or Zune® or Microsoft® Windows devices available from Microsoft of Redmond, Wash. As known in the art, digital media players can serve as both an output device for a computer (e.g., outputting music from a sound file or pictures from an image file) and a storage device. In one embodiment, device 138 may be computer 102, yet in other embodiments, computer 102 may be entirely distinct from device 138. Regardless of whether device 138 is configured to provide certain output, it may serve as an input device for receiving sensory information. Devices 138, 140, 142, 144, and/or 182 may include one or more sensors, including but not limited to: an accelerometer, a gyroscope, a location-determining device (e.g., GPS), light sensor, temperature sensor (including ambient temperature and/or body temperature), heart rate monitor, image-capturing sensor, moisture sensor and/or combinations thereof. In certain embodiments, sensors may be passive, such as reflective materials that may be detected by image-capturing device 126 and/or sensor 128 (among others). In certain embodiments, sensors 144 may be integrated into apparel, such as athletic clothing. For instance, the user 124 may wear one or more on-body sensors 144a-b. Sensors 144 may be incorporated into the clothing of user 124 and/or placed at any desired location of the body of user 124. Sensors 144 may communicate (e.g., wirelessly) with computer 102, sensors 128, 138, 140, and 142, and/or camera 126. Examples of interactive gaming apparel are described in U.S. patent application Ser. No. 10/286,396, filed Oct. 30, 2002, and published as U.S. Pat. Pub. No. 2004/0087366, the contents of which are incorporated herein by reference in its entirety for any and all non-limiting purposes. In certain embodiments, passive sensing surfaces may reflect waveforms, such as infrared light, emitted by image-capturing device 126 and/or sensor 128. In one embodiment, passive sensors located on user's 124 apparel may comprise generally spherical structures made of glass or other transparent or translucent surfaces which may reflect waveforms. Different classes of apparel may be utilized in which a given class of apparel has specific sensors configured to be located proximate to a specific portion of the user's 124 body when properly worn. For example, golf apparel may include one or more sensors positioned on the apparel in a first configuration and yet soccer apparel may include one or more sensors positioned on apparel in a second configuration. Devices 138-144 and 182 may communicate with each other, either directly or through a network, such as network 132. Communication between one or more of devices 138-144 and 182 may communicate through computer 102. For example, two or more of devices 138-144 and 182 may be peripherals operatively connected to bus 114 of computer 102. In yet another embodiment, a first device, such as device 138 may communicate with a first computer, such as computer 102 as well as another device, such as device 142, however, device 142 may not be configured to connect to computer 102 but may communicate with device 138. Those skilled in the art will appreciate that other configurations are possible.

In one embodiment, device 182 may include glasses or eyewear 182. Glasses 182 may be capable of communicating with a user by overlaying visual information onto the lenses of glasses 182. The overlaid information may be placed in a particular region of users' 124 field of vision so as not to interfere or distract user 124. In addition, glasses 182 may also be used to provide audio information to user 124. In yet another embodiment, glasses 182 may also be used as an input device for receiving sensory information from user 124.

Some implementations of the example embodiments may alternately or additionally employ computing devices that are intended to be capable of a wide variety of functions, such as a desktop or laptop personal computer. These computing devices may have any combination of peripheral devices or additional components as desired. Also, the components shown in FIG. 1B may be included in the server 134, other computers, apparatuses, etc.

In certain embodiments, sensory devices 138, 140, 142, 144 and/or 182 may be formed within or otherwise associated with user's 124 clothing or accessories, including a watch, sunglasses, eyeglasses, armband, wristband, necklace, shirt, shoe, or the like. Examples of shoe-mounted and wearable devices are described immediately below, however, these are merely example embodiments and this disclosure should not be limited to such.

In an embodiment, devices such as device 138, 140, 142, 144 and/or 182 may include similar hardware such as the hardware discussed above with respect to computer 102 and in particular the hardware shown in FIG. 1B. For instance, devices 138, 140, 142, 144 and/or 182 may include a processing unit, memory, a CD-ROM, digital versatile disks (DVD) or other optical disk storage, magnetic storage devices, or any other medium that can be used to store the desired information and that can be accessed by computer 102. In addition, devices 138, 140, 142, 144 and/or 182 may also include one or more input devices and one or more output devices. The output devices may include, for example, a monitor display, television, printer, stereo, or speakers. The input devices may include, for example, a keyboard, touch screen, a remote control pad, a pointing device (such as a mouse, touchpad, stylus, trackball, or joystick), a scanner, a camera or a microphone. In this regard, input devices may comprise one or more sensors configured to sense, detect, and/or measure athletic movement from a user, such as user 124, shown in FIG. 1A.

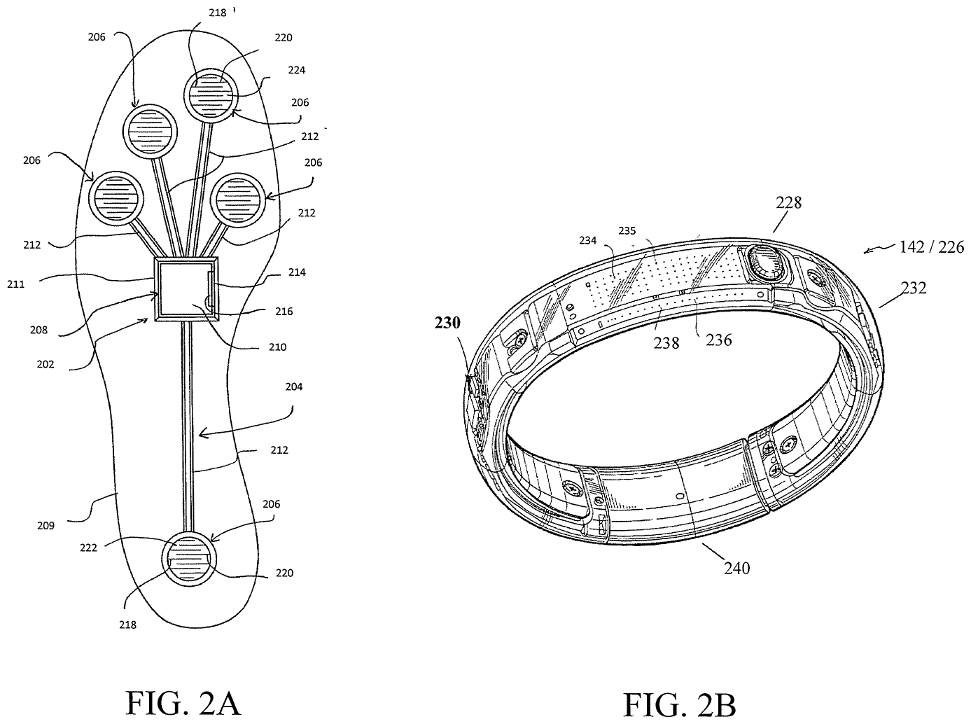

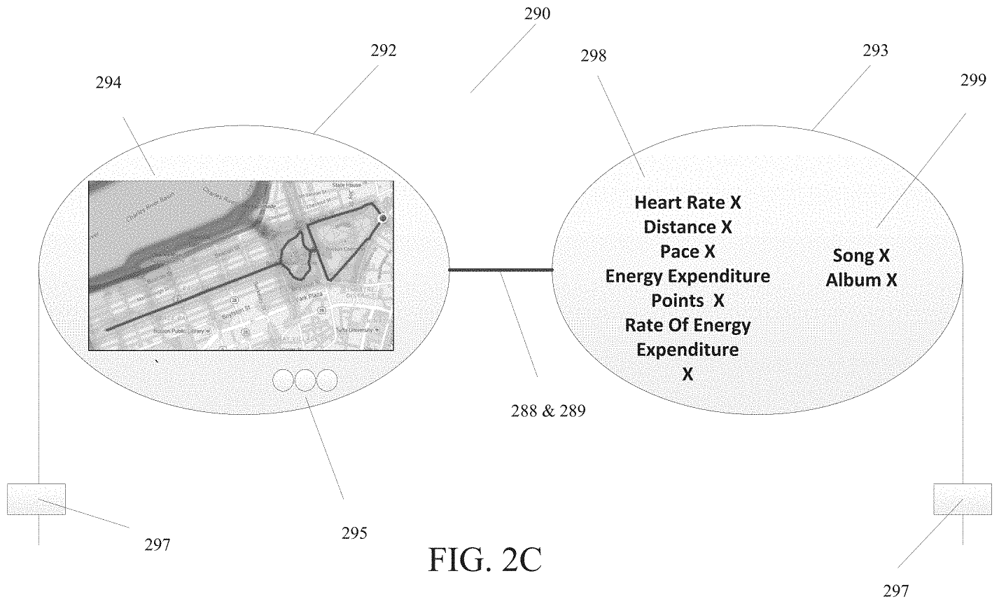

In certain embodiments, sensory device 140 may comprise footwear which may include one or more sensors, including but not limited to: an accelerometer, location-sensing components, such as GPS, and/or a force sensor system. FIG. 2A illustrates one exemplary embodiment of an example sensor system 202. In certain embodiments, system 202 may include a sensor assembly 204. Assembly 204 may comprise one or more sensors, such as for example, an accelerometer, location-determining components, and/or force sensors. In the illustrated embodiment, assembly 204 incorporates a plurality of sensors, which may include force-sensitive resistor (FSR) sensors 206. In yet other embodiments, other sensor(s) may be utilized. Port 208 may be positioned within a sole structure 209 of a shoe. Port 208 may optionally be provided to be in communication with an electronic module 210 (which maybe in a housing 211) and a plurality of leads 212 connecting the FSR sensors 206 to the port 208. Module 210 may be contained within a well or cavity in a sole structure of a shoe. The port 208 and the module 210 include complementary interfaces 214, 216 for connection and communication.

In certain embodiments, at least one force-sensitive resistor 206 shown in FIG. 2A may contain first and second electrodes or electrical contacts 218, 220 and a force-sensitive resistive material 222 and/or 224 disposed between the electrodes 218, 220 to electrically connect the electrodes 218, 220 together. When pressure is applied to the force-sensitive material 222/224, the resistivity and/or conductivity of the force-sensitive material 222/224 changes, which changes the electrical potential between the electrodes 218, 220. The change in resistance can be detected by the sensor system 202 to detect the force applied on the sensor 216. The force-sensitive resistive material 222/224 may change its resistance under pressure in a variety of ways. For example, the force-sensitive material 222/224 may have an internal resistance that decreases when the material is compressed, similar to the quantum tunneling composites described in greater detail below. Further compression of this material may further decrease the resistance, allowing quantitative measurements, as well as binary (on/off) measurements. In some circumstances, this type of force-sensitive resistive behavior may be described as “volume-based resistance,” and materials exhibiting this behavior may be referred to as “smart materials.” As another example, the material 222/224 may change the resistance by changing the degree of surface-to-surface contact. This can be achieved in several ways, such as by using microprojections on the surface that raise the surface resistance in an uncompressed condition, where the surface resistance decreases when the microprojections are compressed, or by using a flexible electrode that can be deformed to create increased surface-to-surface contact with another electrode. This surface resistance may be the resistance between the material 222 and the electrode 218, 220 and/or the surface resistance between a conducting layer (e.g. carbon/graphite) and a force-sensitive layer (e.g. a semiconductor) of a multi-layer material 222/224. The greater the compression, the greater the surface-to-surface contact, resulting in lower resistance and enabling quantitative measurement. In some circumstances, this type of force-sensitive resistive behavior may be described as “contact-based resistance.” It is understood that the force-sensitive resistive material 222/224, as defined herein, may be or include a doped or non-doped semiconducting material.

The electrodes 218, 220 of the FSR sensor 206 can be formed of any conductive material, including metals, carbon/graphite fibers or composites, other conductive composites, conductive polymers or polymers containing a conductive material, conductive ceramics, doped semiconductors, or any other conductive material. The leads 212 can be connected to the electrodes 218, 220 by any suitable method, including welding, soldering, brazing, adhesively joining, fasteners, or any other integral or non-integral joining method. Alternately, the electrode 218, 220 and associated lead(s) 212 may be formed of a single piece of the same material 222/224. In further embodiments, material 222 is configured to have at least one electric property (e.g., conductivity, resistance, etc.) than material 224. Examples of exemplary sensors are disclosed in U.S. patent application Ser. No. 12/483,824, filed on Jun. 12, 2009, the contents of which are incorporated herein in their entirety for any and all non-limiting purposes.

As shown in FIG. 2B, device 226 (which may be, or be a duplicative of or resemble sensory device 142 shown in FIG. 1A) may be configured to be worn by user 124, such as around a wrist, arm, ankle or the like. Device 226 may monitor movements of a user, including, e.g., athletic movements or other activity of user 124. For example, in one embodiment, device 226 may be activity monitor that measures, monitors, tracks or otherwise senses the user's activity (or inactivity) regardless of the user's proximity or interactions with computer 102. Device 226 may detect athletic movement or other activity (or inactivity) during user's 124 interactions with computer 102 and/or operate independently of computer 102. Device 226 may communicate directly or indirectly, wired or wirelessly, with network 132 and/or other devices, such as devices 138 and/or 140. Athletic data obtained from device 226 may be utilized in determinations conducted by computer 102, such as determinations relating to which exercise programs are presented to user 124. As used herein, athletic data means data regarding or relating to a user's activity (or inactivity). In one embodiment, device 226 may wirelessly interact with a remote website such as a site dedicated to fitness or health related subject matter, either directly or indirectly (e.g., via a mobile device, such as device 138 associated with user 124). In this or another embodiment, device 226 may interact with a mobile device, such as device 138, as to an application dedicated to fitness or health related subject matter. In these or other embodiments, device 226 may interest with both a mobile device as to an application as above, such as device 138, and a remote website, such as a site dedicated to fitness or health related subject matter, either directly or indirectly (e.g., via the mobile device, such as device 138). In some embodiments, at some predetermined time(s), the user may wish to transfer data from the device 226 to another location. For example, a user may wish to upload data from a portable device with a relatively smaller memory to a larger device with a larger quantity of memory. Communication between device 226 and other devices may be done wirelessly and/or through wired mechanisms.

As shown in FIG. 2B, device 226 may include an input mechanism, such as a button 228, to assist in operation of the device 226. The button 228 may be a depressible input operably connected to a controller 230 and/or any other electronic components, such as one or more elements of the type(s) discussed in relation to computer 102 shown in FIG. 1B. Controller 230 may be embedded or otherwise part of housing 232. Housing 232 may be formed of one or more materials, including elastomeric components and comprise one or more displays, such as display 234. The display may be considered an illuminable portion of the device 226. The display 234 may include a series of individual lighting elements or light members such as LED lights 234 in an exemplary embodiment. The LED lights may be formed in an array and operably connected to the controller 230. Device 226 may include an indicator system 236, which may also be considered a portion or component of the overall display 234. It is understood that the indicator system 236 can operate and illuminate in conjunction with the display 234 (which may have pixel member 235) or completely separate from the display 234. The indicator system 236 may also include a plurality of additional lighting elements or light members 238, which may also take the form of LED lights in an exemplary embodiment. In certain embodiments, indicator system 236 may provide a visual indication of goals, such as by illuminating a portion of lighting members 238 to represent accomplishment towards one or more goals.

A fastening mechanism 240 can be unlatched wherein the device 226 can be positioned around a wrist of the user 124 and the fastening mechanism 240 can be subsequently placed in a latched position. The user can wear the device 226 at all times if desired. In one embodiment, fastening mechanism 240 may comprise an interface, including but not limited to a USB port, for operative interaction with computer 102 and/or devices 138, 140, and/or recharging an internal power source.

In certain embodiments, device 226 may comprise a sensor assembly (not shown in FIG. 2B). The sensor assembly may comprise a plurality of different sensors. In an example embodiment, the sensor assembly may comprise or permit operative connection to an accelerometer (including in the form of a multi-axis accelerometer), a gyroscope, a location-determining device (e.g., GPS), light sensor, temperature sensor (including ambient temperature and/or body temperature), heart rate monitor, image-capturing sensor, moisture sensor and/or combinations thereof.. Detected movements or parameters from device's 142 sensor(s), may include (or be used to form) a variety of different parameters, metrics or physiological characteristics including but not limited to speed, distance, steps taken, and energy expenditure such as calories, heart rate and sweat detection. Such parameters may also be expressed in terms of activity points or currency earned by the user based on the activity of the user. Examples of wrist-worn sensors that may be utilized in accordance with various embodiments are disclosed in U.S. patent application Ser. No. 13/287,064, filed on Nov. 1, 2011, the contents of which are incorporated herein in their entirety for any and all non-limiting purposes.

As shown in FIG. 2C, device 290 (which may be, or be a duplicative of or resemble device 182 shown in FIG. 1A) may be configured to be in optical alignment with at least one of the user's eyes, such as being placed on the head of user 124, in the form of glasses, sunglasses or protective eyewear. Device 290 may monitor movements of a user, including, e.g., athletic movements or other activity of user 124. In another embodiment, device 290 may provide visual, tactile, and/or audio information to user 124 during a workout or training session. For example, in one embodiment, device 290 may be an activity monitor that measures, monitors, and tracks or otherwise senses the user's activity (or inactivity) regardless of the user's proximity or interactions with computer 102. Device 290 may detect athletic movement or other activity (or inactivity) during user's 124 interactions with computer 102 and/or operate independently of computer 102. Device 290 may communicate directly or indirectly, wired or wirelessly, with network 132 and/or other devices, such as devices 138 and/or 140. Athletic data obtained from device 290 may be utilized in determinations conducted by computer 102, such as determinations relating to which exercise programs are presented to user 124. As used herein, athletic data means data regarding or relating to a user's activity (or inactivity). In one embodiment, device 290 may wirelessly interact with a remote web site such as a site dedicated to fitness or health related subject matter, either directly or indirectly (e.g., via a mobile device, such as device 138 associated with user 124). In this or another embodiment, device 290 may interact with a mobile device, such as device 138, as to an application dedicated to fitness or health related subject matter. In these or other embodiments, device 290 may interest with both a mobile device as to an application as above, such as device 138, and a remote website, such as a site dedicated to fitness or health related subject matter, either directly or indirectly (e.g., via the mobile device, such as device 138). In some embodiments, at some predetermined time(s), the user may wish to transfer data from the device 290 to another location. For example, a user may wish to upload data from a portable device with a relatively smaller memory to a larger device with a larger quantity of memory. Communication between device 290 and other devices may be done wirelessly and/or through wired mechanisms.

In an embodiment, device 290 may display on lenses 292 and 293 information useful to user 124 during a workout or training session. Such information may include a top route such as top route 294 or other geographical information related to a run or cycling session. Such routing information may also display a user's progress on the top route as the user proceeds along the route. During the workout, device 290 may include alternative routes which may alter (e.g., increase or decrease) the intensity of the overall workout session. Such alternative routes may include grade or elevation changes to make the workout more difficult and assist user 124 in obtaining their workout goals for the workout session. In addition, device 124 may suggest alternative routes which user 124 has not taken before to motivate user 124 during the running session with new scenery to be viewed during the workout. Such alternative route determinations by device 290 may be based on a user's workout preferences, fitness needs, and implicit security requirements. In an embodiment, user 124 may be delighted to have route recommendations provided that indicate which of their friends or favorite athletes have run the route, sightseeing opportunities for the new route, as well as areas of interests for user 124. User 124 may also be provided with information through device 290 that suites their fitness needs such as improved endurance or builds strength and speed. For instance, information such as information 298 may be shown on lens 293 during a workout. Such information may include heart rate monitoring, distance, pace, and energy expenditure points or score, along with other workout statistics. In addition, information related to other devices associated with user 124 may also optionally be displayed such as song information 299 as shown in lens 293.

In an embodiment, device 290 may also provide and utilize real-time information based on construction, traffic reports, and safety events in areas (e.g. Police Actions) and provide detours or route alternatives when needed. In an embodiment, device 290 may also detect and alert user 124 to potential dangers such as a predicted impact with an oncoming car or pedestrian. In another embodiment, device 290 may at various points during a run such as at intersections display to user 124 alternative routes along with the benefits of taking the alternative route (i.e. longer/shorter distance, more energy expenditure points, elevation changes, scenic route etc.).

As shown in FIG. 2C, device 290 may include lenses 292 and 293 and speakers 297. A controller 288 and associated memory 289 may be embedded or otherwise part of glasses 290. In an embodiment, information on lenses 292 and 293 may be projected onto the lenses by a micro-projector. The micro-projector may display information or shapes into a user's field of vision. In another embodiment, lenses 292 and 293 may include a series of individual lighting elements or light members such as LED lights. The LED lights may be formed in an array and operably connected to the controller 288. Device 290 may include an indicator system 295, which may also be considered a portion or component of the overall display. It is understood that indicator system 295 can operate and illuminate in conjunction with the display shown on lenses 292 and 293. The indicator system 295 may also include a plurality of additional lighting elements or light members, which may also take the form of LED lights in an exemplary embodiment. In certain embodiments, indicator system 295 may provide a visual indication of goals, such as by illuminating to represent accomplishment towards one or more goals. In an embodiment, the indicator system 295 may inform user 124 of their progress in achieving a goal by overlaying visual channel information with energy expenditure symbols that change color from red to green as user 124 gets closer to reaching their workout goal. In an embodiment, the size of the displayed symbol may be used to communicate information to user 124 such as an elevation increases during a run. In addition, milestones may be communicated, such as distance markers. In another embodiment, indicator system 295 may also be used to designate and ideal running route for user 124 during a workout.

In certain embodiments, device 290 may comprise a sensor assembly (not shown in FIG. 2C). The sensor assembly may comprise a plurality of different sensors. In an example embodiment, the sensor assembly may comprise or permit operative connection to an accelerometer (including in the form of a multi-axis accelerometer), a gyroscope, a location-determining device (e.g., GPS), light sensor, temperature sensor (including ambient temperature and/or body temperature), heart rate monitor, image-capturing sensor, moisture sensor and/or combinations thereof. Detected movements or parameters from device's sensor(s), may include (or be used to form) a variety of different parameters, metrics or physiological characteristics including but not limited to speed, distance, steps taken, and energy expenditure such as calories, heart rate and sweat detection. Such parameters may also be expressed in terms of activity points or currency earned by the user based on the activity of the user.

In an embodiment, system 100 may prompt a user to perform one or more exercises, monitor user movement while performing the exercises, and provide the user with feedback based on their performance. In one embodiment, computer 102, image-capturing device 126, sensor 128, and display 136 may be implemented within the confines of a user's residence, although other locations, including schools, gyms and/or businesses are contemplated. Further, as discussed above, computer 102 may be a portable device, such as a cellular telephone, therefore, one or more aspects discussed herein may be conducted in almost any location.

While exercising, system 100 may use one or more techniques to monitor user movement. The method may be implemented by a computer, such as, for example, computer 102, device 138, 140, 142, 144, 182, and/or other apparatuses.

In an embodiment, system 100 may process sensory data to identify user movement data. In one embodiment, sensory locations may be identified. For example, images of recorded video, such as from image-capturing device 126, may be utilized in an identification of user movement. For example, the user may stand a certain distance, which may or may not be predefined, from the image-capturing device 126, and computer 102 may process the images to identify the user 124 within the video, for example, using disparity mapping techniques. In an example, the image capturing device 126 may be a stereo camera having two or more lenses that are spatially offset from one another and that simultaneously capture two or more images of the user. Computer 102 may process the two or more images taken at a same time instant to generate a disparity map for determining a location of certain parts of the user's body in each image (or at least some of the images) in the video using a coordinate system (e.g., Cartesian coordinates). The disparity map may indicate a difference between images taken by each of the offset lenses.

In a second example, one or more sensors may be located on or proximate to the user's 124 body at various locations or wear a suit having sensors situated at various locations. Yet, in other embodiments, sensor locations may be determined from other sensory devices, such as devices 138, 140, 142, 144 and/or 182. With reference to FIG. 4, sensors may be placed (or associated with, such as with image-capturing device 126) body movement regions, such as joints (e.g., ankles, elbows, shoulders, etc.) or at other locations of interest on the user's 124 body. Example sensory locations are denoted in FIG. 4 by locations 402a-402o. In this regard, sensors may be physical sensors located on/in a user's clothing, yet in other embodiments, sensor locations 402a-402o may be based upon identification of relationships between two moving body parts. For example, sensor location 402a may be determined by identifying motions of user 124 with an image-capturing device, such as image-capturing device 126. Thus, in certain embodiments, a sensor may not physically be located at a specific location (such as sensor locations 402a-402o), but is configured to sense properties of that location, such as with image-capturing device 126. In this regard, the overall shape or portion of a user's body may permit identification of certain body parts. Regardless of whether an image-capturing device, such as camera 126, is utilized and/or a physical sensor located on the user 124, such as sensors within or separate from one or more of device(s) 138, 140, 142, 144 are utilized, the sensors may sense a current location of a body part and/or track movement of the body part. In one embodiment, location 402m may be utilized in a determination of the user's center of gravity (a.k.a, center of mass).

For example, relationships between location 402a and location(s) 402f/402l with respect to one or more of location(s) 402m-402o may be utilized to determine if a user's center of gravity has been elevated along the vertical axis (such as during a jump) or if a user is attempting to “fake” a jump by bending and flexing their knees. In one embodiment, sensor location 402n may be located at about the sternum of user 124. Likewise, sensor location 402o may be located approximate to the naval of user 124. In certain embodiments, data from sensor locations 402m-402o may be utilized (alone or in combination with other data) to determine the center of gravity for user 124. In further embodiments, relationships between multiple several sensor locations, such as sensors 402m-402o, may be utilized in determining orientation of the user 124 and/or rotational forces, such as twisting of user's 124 torso. Further, one or more locations, such as location(s), may be utilized to as a center of moment location. For example, in one embodiment, one or more of location(s) 402m-402o may serve as a point for a center of moment location of user 124. In another embodiment, one or more locations may serve as a center of moment of specific body parts or regions.

In certain embodiments, a time stamp to the data collected indicating a specific time when a body part was at a certain location. Sensor data may be received at computer 102 (or other device) via wireless or wired transmission. A computer, such as computer 102 and/or devices 138, 140, 142, 144, 182 may process the time stamps to determine the locations of the body parts using a coordinate system (e.g., Cartesian coordinates) within each (or at least some) of the images in the video. Data received from image-capturing device 126 may be corrected, modified, and/or combined with data received from one or more other devices 138, 140, 142, 144 and 182.

In a third example, computer 102 may use infrared pattern recognition to detect user movement and locations of body parts of the user 124. For example, the sensor 128 may include an infrared transceiver, which may be part of image-capturing device 126, or another device, that may emit an infrared signal to illuminate the user's 124 body using infrared signals. The infrared transceiver 128 may capture a reflection of the infrared signal from the body of user 124. Based on the reflection, computer 102 may identify a location of certain parts of the user's body using a coordinate system (e.g., Cartesian coordinates) at particular instances in time. Which and how body parts are identified may be predetermined based on a type of exercise a user is requested to perform.

As part of a workout routine, computer 102 may make an initial postural assessment of the user 124 as part of the initial user assessment. Computer 102 may analyze front and side images of a user 124 to determine a location of one or more of a user's shoulders, upper back, lower back, hips, knees, and ankles. On-body sensors and/or infrared techniques may also be used, either alone or in conjunction with image-capturing device 126, to determine the locations of various body parts for the postural assessment.

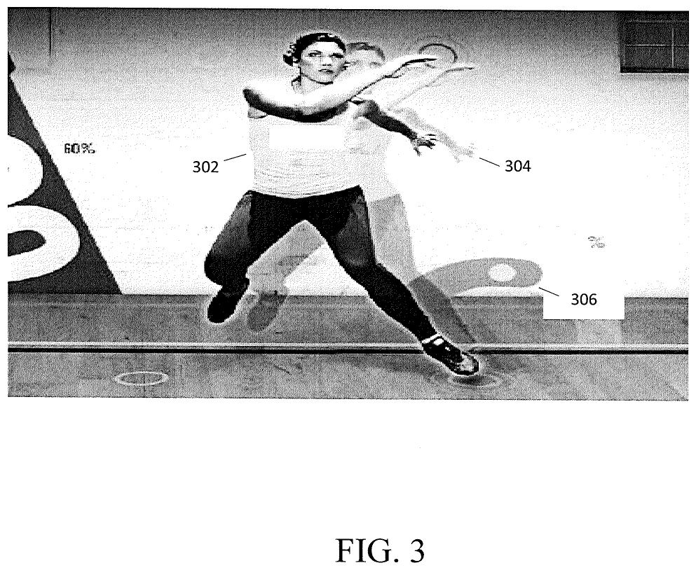

While performing an exercise, computer 102 may cause a display, such as display 136 or device 182, to present a user representation with real-time feedback. While user 124 is performing movements, computer 102 may create a user representation for display by the display 136 or device 182. The computer may create the user representation based on one or more of processing some or all images of video captured by image capturing device 126, processing data received from the sensor 128, and processing data received from sensors 138, 140, 142, 144, and 182. The user representation may be, for example, video of the user, or a user avatar 302 (FIG. 3) created based on image and/or sensor data, including infrared data.

In an embodiment, a user's past workout performance may be stored as a virtual shadow for later playback. In an embodiment, numerous virtual shadows may be stored for a user, each virtual shadow representing a prior exercise performance. In an embodiment, displaying of multiple virtual shadows may allow a user, such as user 124, to see changes in their workout performances.

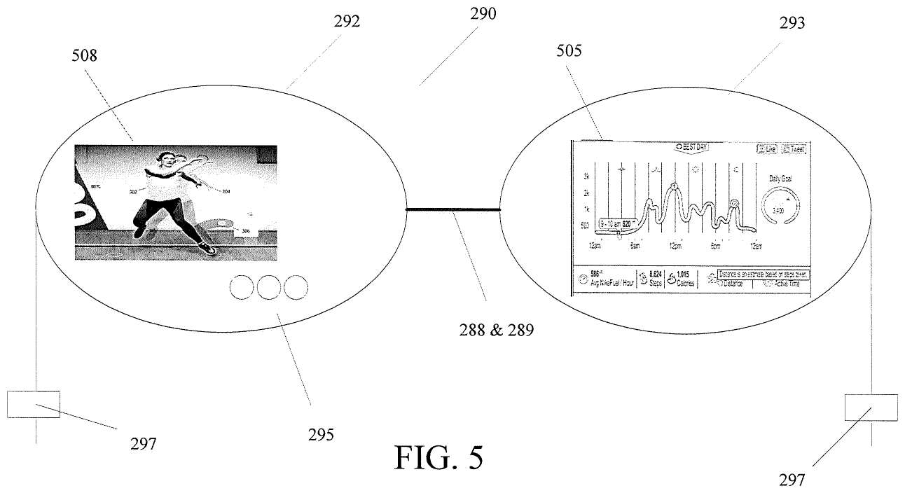

In an embodiment, user avatar 302 may be generated and displayed with the appearance that a user, such as user 124, is competing against themselves. For example, computer 102 (or any other electronic device such as device 182) may generate and store performance information related to a user's completed workout (i.e. virtual shadow). Later, computer 102 may prompt the user if they would like to compete in real-time against their earlier performance of the exercise. In that case, system 100 may display user avatar 302 and stored virtual shadow 304 for the competition. User avatar 302 along with virtual shadow 304 may be displayed as part of a display 508 in glasses 290 as shown in FIG. 5.

In an embodiment, the generated user avatar 302 and virtual shadow 304 may permit a user to view workout improvements over time, including, as examples, the latest improvement or improvement over a (e.g., user-selected) time period or improvement from a beginning.

In another embodiment, a user may compare a past running performance on a particular running route that has numerous elevation changes to a current and different route with minimal elevation changes. The results may assist the user in gauging the user's pace and other metrics in different run settings. In an embodiment, during a run the system may recommend route changes or modifications based on a target goal such as energy expenditure or a rate of energy expenditure.

When competing against him or herself, computer 102 may display user avatar 302 as the user performs an exercise for simultaneous display along with the virtual shadow 304 (i.e. representing prior workout performance information). User avatar 302 may be displayed overtop of or directly behind the virtual shadow 304, as seen in FIG. 3. Alternatively, the display 136 or device 182 may present virtual shadow 304 offset from user avatar 302. Computer 102 may synchronize the start times such that user avatar 302 appears to be competing against virtual shadow 304 in real-time. When an exercise is complete, computer 102 may inform the user 124 of the winner, and provide side by side statistics of the current performance relative to the virtual shadow 304

Display 136 or device 182 may also present one or more performance level indicators 306 to indicate a user's performance metrics, as depicted in FIG. 3. Performance level indicators may be displayed instead of a shadow. Yet, in other embodiments, indicators may be displayed in conjunction with a shadow. Example metrics may include speed, quickness, power, dimensions (e.g., distance stepped or dipped, height jumped, rotation of hips or shoulders), reaction time, agility, flexibility, acceleration, heart rate, temperature (e.g., overheating), blood oxygen content, or other physical or physiological metrics. A performance level indicator 306 may be depicted as, for example, a gauge, a speedometer, a bar-type indictor, percentage indicator, etc. In another embodiment, performance level indicators may also be displayed to a user in a separate portion of the display 505 as shown in FIG. 5 on lens 293.

In another embodiment, a virtual shadow 304 may be displayed with the appearance that a user, such as user 124, is competing against another user. In one embodiment, user 124 may be located at a first physical location and a second user may be located at a second physical location. A location may include a place or a geographical position such as a gym, dwelling, school, or even exercising outside, such as running through a city. Despite being at different physical locations, users may still compete and/or collectively engage in athletic activities. In one embodiment, each of a plurality of users may engage in a competition in substantially real-time. Yet, in other embodiments, a first user may conduct a predefined series of activities or routines and data from that first user's performance may be utilized in a later conducted competition. In one embodiment, two or more users may engage in a “side-by-side” competition. For example, computer 102 (or any other electronic device) may display a user avatar 302 while a first user 124 performs an exercise. The same computer 102 and/or another computer, such as an electronic device that is in operative communication with network 132, may generate and/or store a second avatar representing the second user. Both of these avatars may be displayed on a single display device, such as display 136 or device 182 at the location of user 124 (and/or at the location of the second user). Thus, user 124 may see both avatars.

In an embodiment, virtual shadows may be generated based upon past performances in one or more activities, such as the activity being performed in competition or upon an assessment of a person's respective capabilities (e.g., current fitness level).

In other embodiments, users may compete with another user's virtual shadow. For example, a first user, such as user 124 may have had a great workout and want to challenge a second user to see how they perform or stack up against the first user's past workout. A virtual shadow representing the first user's past workout may be transmitted to permit the second user to compete against the first user's performance. In one embodiment, a user avatar 302 of the second user may be displayed on display 136. A virtual shadow 304 may be generated based upon the workout of the first user 124. System 100 may synchronize the start times such that the user avatar 302 appears to be competing against the virtual shadow 304. When an exercise is complete, computer 102 may inform either user of the winner. System 100 may also provide side by side statistics of the second user's current performance relative to the virtual shadow 304 of the first user 124. Competing with other users' virtual shadow(s) 304 may be performed in a real-time environment as well as permitting virtual shadows 304 from previous athletic activities to be utilized.

In an embodiment, map data or topographical map data may be used as background to show the avatar's location on the route during a workout. During the work out, indicators showing the instantaneous values of various measured time, distance, physical, and/or physiological parameters associated with the athletic performance at locations along the route traveled by the virtual athlete may be displayed.

In an embodiment, a second indicator display region also may be provided to display instantaneous values of various measured time, distance, physical, and/or physiological parameters associated with the virtual athlete athletic performance at locations along the route. Of course, the data for the two athletic performances may be obtained from any source(s) without departing from the invention.

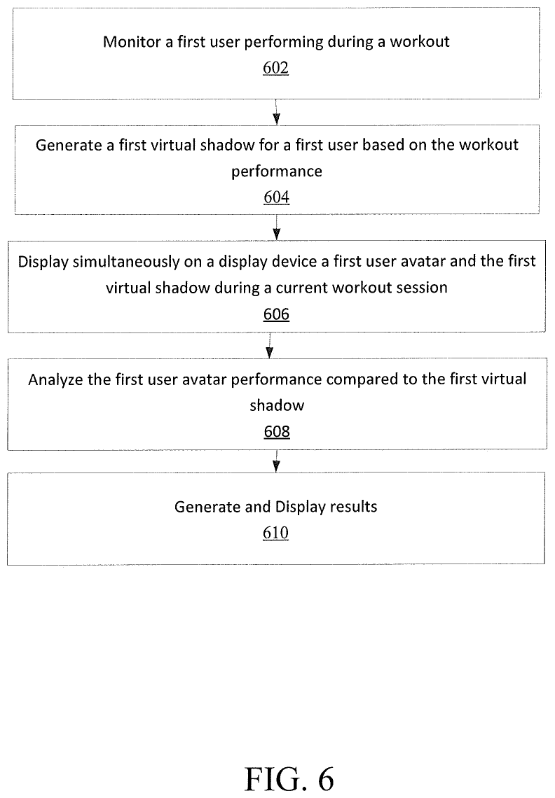

In an embodiment, system 100 may monitor a first user workout as illustrated in step 602 of FIG. 6. In an example, computer 102 may prompt a user to perform one or more exercises during a workout session. A workout session may include a predetermined number of exercises or involve a single athletic activity (e.g., run 10 miles).

In an embodiment, a first user avatar may be generated for user 124. As explained throughout this disclosure, multiple sensors may be utilized, either in combination or alone, to monitor data. In one embodiment, computer 102 may generate a user avatar of the user based on data captured by one or more of sensors 128, 138, 140, 142, 144, 182, and/or camera 126

In step 604, a first virtual shadow for a first user may be generated based on the workout performance monitored in step 602. As part of a new workout session, user 124 may compete against their previous performance or another user. For example, computer 102 may display a first user avatar and a first virtual shadow, where the first user avatar corresponds to the user's current real-time performance, and the first virtual shadow corresponds to a previous performance of the workout session. In step 606, a device such as device 182 may simultaneously display to user 124 a first user avatar and the first virtual shadow during a current real-time workout session.

In another example, a second user may complete a particular workout session where their computer monitors the second user's performance, and cause their computer to send a challenge to computer 102 challenging the first user to beat their performance. The challenge may include data of the second user performing the particular workout session.

In a further example, both users may perform a workout session at the same time, where respective computers 102 may monitor each user's performance, and exchange data with the other user's computer via network 132 so that each computer can cause display of the other's user avatar in a virtual competition.

In step 608, the system 100 may analyze the first user avatar performance compared to the first virtual shadow. In step 610, results may be generated and displayed.

Providing an activity environment having one or more of the features described herein may provide a user with an immersive experience that will encourage and motivate the user to engage in athletic activities and improve his or her fitness. Users may further communicate through social communities and challenge one another to reach various levels of fitness, and to view their fitness level and activity.

Aspects of the embodiments have been described in terms of illustrative embodiments thereof. Numerous other embodiments, modifications and variations within the scope and spirit of the appended claims will occur to persons of ordinary skill in the art from a review of this disclosure. For example, one of ordinary skill in the art will appreciate that the steps illustrated in the illustrative figures may be performed in other than the recited order, and that one or more steps illustrated may be optional in accordance with aspects of the disclosure.

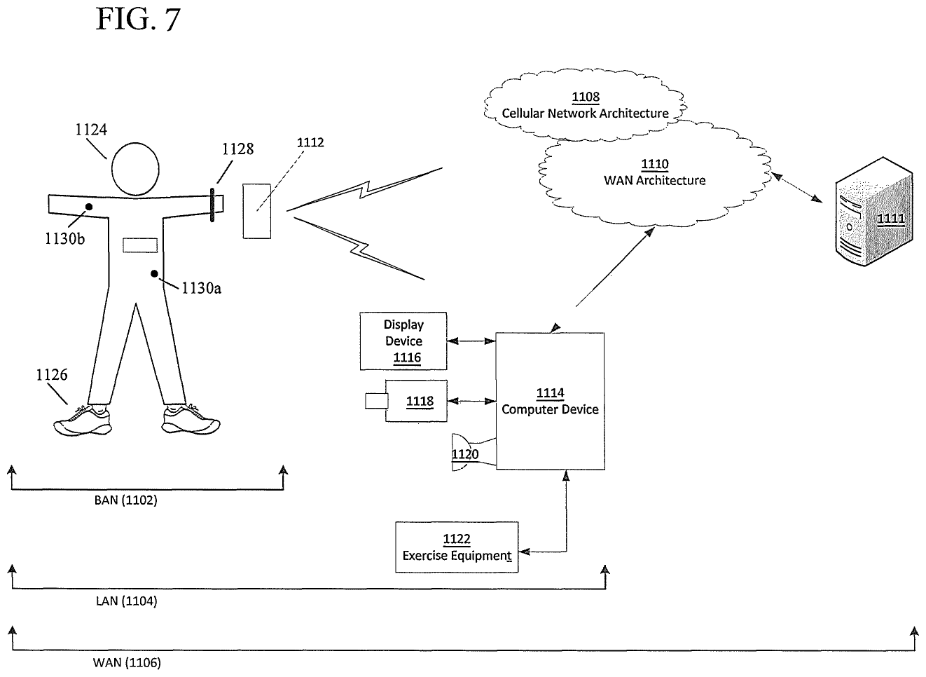

FIGS. 7-10 illustrate another exemplary operating environment which may be used with various aspects of the disclosure. It is to be understood that other embodiments may be utilized and structural and functional modifications may be made without departing from the scope and spirit of the present disclosure. Further, headings within this disclosure should not be considered as limiting aspects of the disclosure and the example embodiments are not limited to the example headings.

### I. Example Personal Training System

A. Illustrative Networks

Aspects of this disclosure relate to systems and methods that may be utilized across a plurality of networks. In this regard, certain embodiments may be configured to adapt to dynamic network environments. Further embodiments may be operable in differing discrete network environments. FIG. 7 illustrates an example of a personal training system 1100 in accordance with example embodiments. Example system 1100 may include one or more interconnected networks, such as the illustrative body area network (BAN) 1102, local area network (LAN) 1104, and wide area network (WAN) 1106. As shown in FIG. 7 (and described throughout this disclosure), one or more networks (e.g., BAN 1102, LAN 1104, and/or WAN 1106), may overlap or otherwise be inclusive of each other. Those skilled in the art will appreciate that the illustrative networks 1102-1106 are logical networks that may each comprise one or more different communication protocols and/or network architectures and yet may be configured to have gateways to each other or other networks. For example, each of BAN 1102, LAN 1104 and/or WAN 1106 may be operatively connected to the same physical network architecture, such as cellular network architecture 1108 and/or WAN architecture 1110. For example, portable electronic device 1112, which may be considered a component of both BAN 1102 and LAN 1104, may comprise a network adapter or network interface card (NIC) configured to translate data and control signals into and from network messages according to one or more communication protocols, such as the Transmission Control Protocol (TCP), the Internet Protocol (IP), and the User Datagram Protocol (UDP) through one or more of architectures 1108 and/or 1110. These protocols are well known in the art, and thus will not be discussed here in more detail.

Network architectures 1108 and 1110 may include one or more information distribution network(s), of any type(s) or topology(s), alone or in combination(s), such as for example, cable, fiber, satellite, telephone, cellular, wireless, etc. and as such, may be variously configured such as having one or more wired or wireless communication channels (including but not limited to: WiFi®, Bluetooth®, Near-Field Communication (NFC) and/or ANT technologies). Thus, any device within a network of FIG. 7, (such as portable electronic device 1112 or any other device described herein) may be considered inclusive to one or more of the different logical networks 1102-1106. With the foregoing in mind, example components of an illustrative BAN and LAN (which may be coupled to WAN 1106) will be described.

1. Example Local Area Network

LAN 1104 may include one or more electronic devices, such as for example, computer device 1114. Computer device 1114, or any other component of system 1100, may comprise a mobile terminal, such as a telephone, music player, tablet, netbook or any portable device. In other embodiments, computer device 1114 may comprise a media player or recorder, desktop computer, server(s), a gaming console, such as for example, a Microsoft® XBOX, Sony® Playstation, and/or a Nintendo® Wii gaming consoles. Those skilled in the art will appreciate that these are merely example devices for descriptive purposes and this disclosure is not limited to any console or computing device.

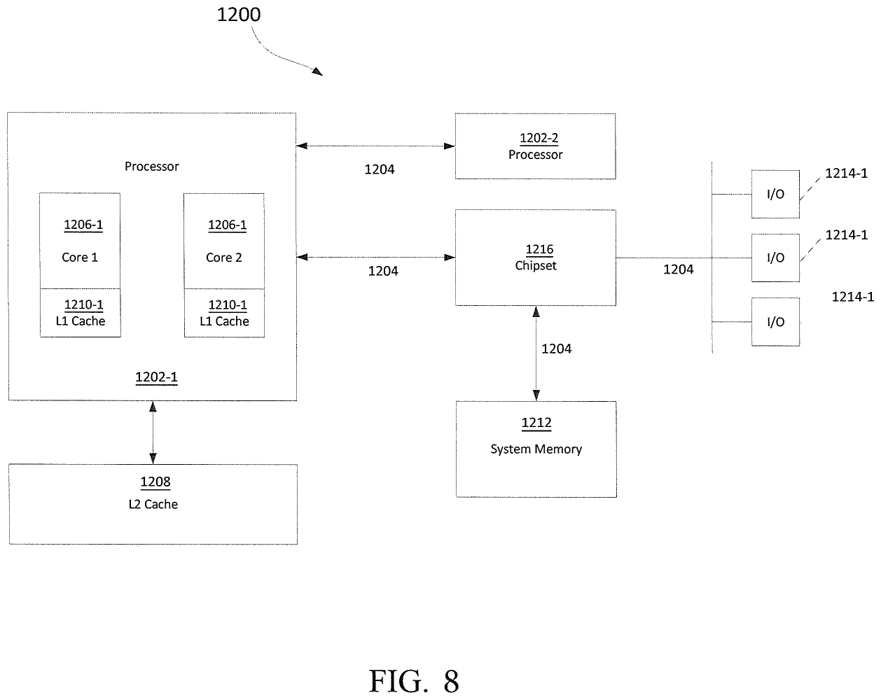

Those skilled in the art will appreciate that the design and structure of computer device 1114 may vary depending on several factors, such as its intended purpose. One example implementation of computer device 1114 is provided in FIG. 8, which illustrates a block diagram of computing device 1200. Those skilled in the art will appreciate that the disclosure of FIG. 8 may be applicable to any device disclosed herein. Device 1200 may include one or more processors, such as processor 1202-1 and 1202-2 (generally referred to herein as “processors 1202” or “processor 1202”). Processors 1202 may communicate with each other or other components via an interconnection network or bus 1204. Processor 1202 may include one or more processing cores, such as cores 1206-1 and 1206-2 (referred to herein as “cores 1206” or more generally as “core 1206”), which may be implemented on a single integrated circuit (IC) chip.

Cores 1206 may comprise a shared cache 1208 and/or a private cache (e.g., caches 1210-1 and 1210-2, respectively). One or more caches 1208/1210 may locally cache data stored in a system memory, such as memory 1212, for faster access by components of the processor 1202. Memory 1212 may be in communication with the processors 1202 via a chipset 1216. Cache 1208 may be part of system memory 1212 in certain embodiments. Memory 1212 may include, but is not limited to, random access memory (RAM), read only memory (ROM), and include one or more of solid-state memory, optical or magnetic storage, and/or any other medium that can be used to store electronic information. Yet other embodiments may omit system memory 1212.

System 1200 may include one or more I/O devices (e.g., I/O devices 1214-1 through 12143, each generally referred to as I/O device 1214). I/O data from one or more I/O devices 1214 may be stored at one or more caches 1208, 1210 and/or system memory 1212. Each of I/O devices 1214 may be permanently or temporarily configured to be in operative communication with a component of system 1100 using any physical or wireless communication protocol.

Returning to FIG. 7, four example I/O devices (shown as elements 1116-1122) are shown as being in communication with computer device 1114. Those skilled in the art will appreciate that one or more of devices 1116-1122 may be stand-alone devices or may be associated with another device besides computer device 1114. For example, one or more I/O devices may be associated with or interact with a component of BAN 1102 and/or WAN 1106. I/O devices 1116-1122 may include, but are not limited to athletic data acquisition units, such as for example, sensors. One or more I/O devices may be configured to sense, detect, and/or measure an athletic parameter from a user, such as user 1124. Examples include, but are not limited to: an accelerometer, a gyroscope, a location-determining device (e.g., GPS), light (including non-visible light) sensor, temperature sensor (including ambient temperature and/or body temperature), sleep pattern sensors, heart rate monitor, image-capturing sensor, moisture sensor, force sensor, compass, angular rate sensor, and/or combinations thereof among others.

In further embodiments, I/O devices 1116-1122 may be used to provide an output (e.g., audible, visual, or tactile cue) and/or receive an input, such as a user input from athlete 1124. Example uses for these illustrative I/O devices are provided below, however, those skilled in the art will appreciate that such discussions are merely descriptive of some of the many options within the scope of this disclosure. Further, reference to any data acquisition unit, I/O device, or sensor is to be interpreted disclosing an embodiment that may have one or more I/O device, data acquisition unit, and/or sensor disclosed herein or known in the art (either individually or in combination).

Information from one or more devices (across one or more networks) may be used to provide (or be utilized in the formation of) a variety of different parameters, metrics or physiological characteristics including but not limited to: motion parameters, such as speed, acceleration, distance, steps taken, direction, relative movement of certain body portions or objects to others, or other motion parameters which may be expressed as angular rates, rectilinear rates or combinations thereof, physiological parameters, such as calories, heart rate, sweat detection, effort, oxygen consumed, oxygen kinetics, and other metrics which may fall within one or more categories, such as: pressure, impact forces, information regarding the athlete, such as height, weight, age, demographic information and combinations thereof.

System 1100 may be configured to transmit and/or receive athletic data, including the parameters, metrics, or physiological characteristics collected within system 1100 or otherwise provided to system 1100. As one example, WAN 1106 may comprise server 1111. Server 1111 may have one or more components of system 1200 of FIG. 8. In one embodiment, server 1111 comprises at least a processor and a memory, such as processor 1206 and memory 1212. Server 1111 may be configured to store computer-executable instructions on a non-transitory computer-readable medium. The instructions may comprise athletic data, such as raw or processed data collected within system 1100. System 1100 may be configured to transmit data, such as energy expenditure points, to a social networking website or host such a site. Server 1111 may be utilized to permit one or more users to access and/or compare athletic data. As such, server 1111 may be configured to transmit and/or receive notifications based upon athletic data or other information.

Returning to LAN 1104, computer device 1114 is shown in operative communication with a display device 1116, an image-capturing device 1118, sensor 1120 and exercise device 1122, which are discussed in turn below with reference to example embodiments. In one embodiment, display device 1116 may provide audio-visual cues to athlete 1124 to perform a specific athletic movement. The audio-visual cues may be provided in response to computer-executable instruction executed on computer device 1114 or any other device, including a device of BAN 1102 and/or WAN. Display device 1116 may be a touchscreen device or otherwise configured to receive a user-input.

In one embodiment, data may be obtained from image-capturing device 1118 and/or other sensors, such as sensor 1120, which may be used to detect (and/or measure) athletic parameters, either alone or in combination with other devices, or stored information. Image-capturing device 1118 and/or sensor 1120 may comprise a transceiver device. In one embodiment sensor 1128 may comprise an infrared (IR), electromagnetic (EM) or acoustic transceiver. For example, image-capturing device 1118, and/or sensor 1120 may transmit waveforms into the environment, including towards the direction of athlete 1124 and receive a “reflection” or otherwise detect alterations of those released waveforms. Those skilled in the art will readily appreciate that signals corresponding to a multitude of different data spectrums may be utilized in accordance with various embodiments. In this regard, devices 1118 and/or 1120 may detect waveforms emitted from external sources (e.g., not system 100). For example, devices 1118 and/or 1120 may detect heat being emitted from user 1124 and/or the surrounding environment. Thus, image-capturing device 1126 and/or sensor 1128 may comprise one or more thermal imaging devices. In one embodiment, image-capturing device 1126 and/or sensor 1128 may comprise an IR device configured to perform range phenomenology.

In one embodiment, exercise device 1122 may be any device configurable to permit or facilitate the athlete 1124 performing a physical movement, such as for example a treadmill, step machine, etc. There is no requirement that the device be stationary. In this regard, wireless technologies permit portable devices to be utilized, thus a bicycle or other mobile exercising device may be utilized in accordance with certain embodiments. Those skilled in the art will appreciate that equipment 1122 may be or comprise an interface for receiving an electronic device containing athletic data performed remotely from computer device 1114. For example, a user may use a sporting device (described below in relation to BAN 1102) and upon returning home or the location of equipment 1122, download athletic data into element 1122 or any other device of system 1100. Any I/O device disclosed herein may be configured to receive activity data.

2. Body Area Network

BAN 1102 may include two or more devices configured to receive, transmit, or otherwise facilitate the collection of athletic data (including passive devices). Exemplary devices may include one or more data acquisition units, sensors, or devices known in the art or disclosed herein, including but not limited to I/O devices 1116-1122. Two or more components of BAN 1102 may communicate directly, yet in other embodiments, communication may be conducted via a third device, which may be part of BAN 1102, LAN 1104, and/or WAN 1106. One or more components of LAN 1104 or WAN 1106 may form part of BAN 1102. In certain implementations, whether a device, such as portable device 1112, is part of BAN 1102, LAN 1104, and/or WAN 1106, may depend on the athlete's proximity to an access point to permit communication with mobile cellular network architecture 108 and/or WAN architecture 1110. User activity and/or preference may also influence whether one or more components are utilized as part of BAN 1102. Example embodiments are provided below.

User 1124 may be associated with (e.g., possess, carry, wear, and/or interact with) any number of devices, such as portable device 1112, shoe-mounted device 1126, wrist-worn device 1128 and/or a sensing location, such as sensing location 1130, which may comprise a physical device or a location that is used to collect information. One or more devices 1112, 1126, 1128, and/or 1130 may not be specially designed for fitness or athletic purposes. Indeed, aspects of this disclosure relate to utilizing data from a plurality of devices, some of which are not fitness devices, to collect, detect, and/or measure athletic data. In certain embodiments, one or more devices of BAN 1102 (or any other network) may comprise a fitness or sporting device that is specifically designed for a particular sporting use. As used herein, the term “sporting device” includes any physical object that may be used or implicated during a specific sport or fitness activity. Exemplary sporting devices may include, but are not limited to: golf balls, basketballs, baseballs, soccer balls, footballs, powerballs, hockey pucks, weights, bats, clubs, sticks, paddles, mats, and combinations thereof. In further embodiments, exemplary fitness devices may include objects within a sporting environment where a specific sport occurs, including the environment itself, such as a goal net, hoop, backboard, portions of a field, such as a midline, outer boundary marker, base, and combinations thereof.

In this regard, those skilled in the art will appreciate that one or more sporting devices may also be part of (or form) a structure and vice-versa, a structure may comprise one or more sporting devices or be configured to interact with a sporting device. For example, a first structure may comprise a basketball hoop and a backboard, which may be removable and replaced with a goal post. In this regard, one or more sporting devices may comprise one or more sensors, such as one or more of the sensors discussed above in relation to FIGS. 7-9, that may provide information utilized, either independently or in conjunction with other sensors, such as one or more sensors associated with one or more structures. For example, a backboard may comprise a first sensor configured to measure a force and a direction of the force by a basketball upon the backboard and the hoop may comprise a second sensor to detect a force. Similarly, a golf club may comprise a first sensor configured to detect grip attributes on the shaft and a second sensor configured to measure impact with a golf ball.

Looking to the illustrative portable device 1112, it may be a multi-purpose electronic device, that for example, includes a telephone or digital music player, including an IPOD®, IPAD®, or iPhone®, brand devices available from Apple, Inc. of Cupertino, Calif. or Zune® or Microsoft® Windows devices available from Microsoft of Redmond, Wash. As known in the art, digital media players can serve as an output device, input device, and/or storage device for a computer. Device 1112 may be configured as an input device for receiving raw or processed data collected from one or more devices in BAN 1102, LAN 1104, or WAN 1106. In one or more embodiments, portable device 1112 may comprise one or more components of computer device 1114. For example, portable device 1112 may be include a display 1116, image-capturing device 1118, and/or one or more data acquisition devices, such as any of the I/O devices 11161122 discussed above, with or without additional components, so as to comprise a mobile terminal.

a. Illustrative Apparel/Accessory Sensors

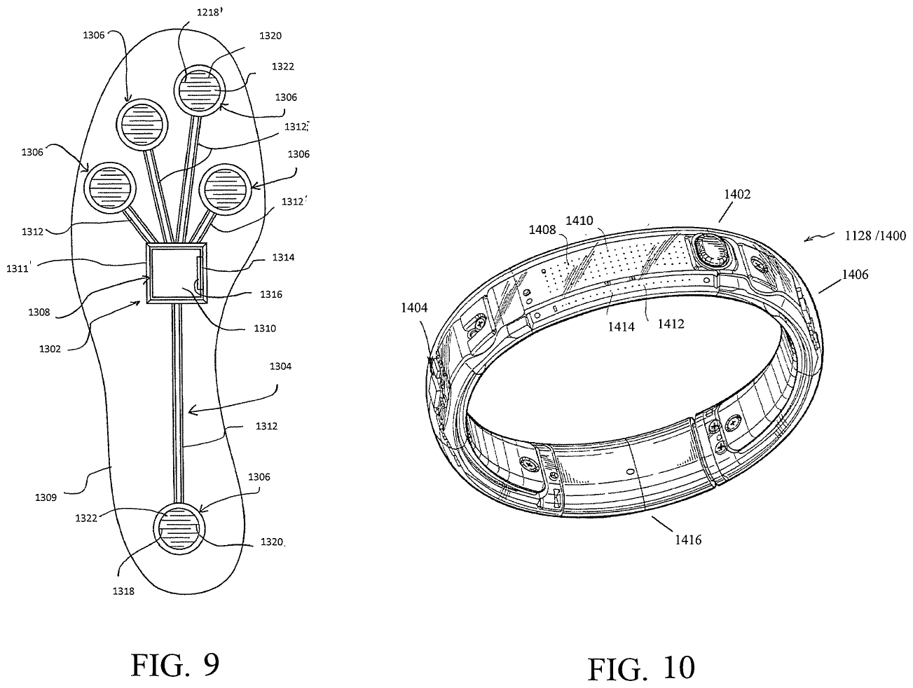

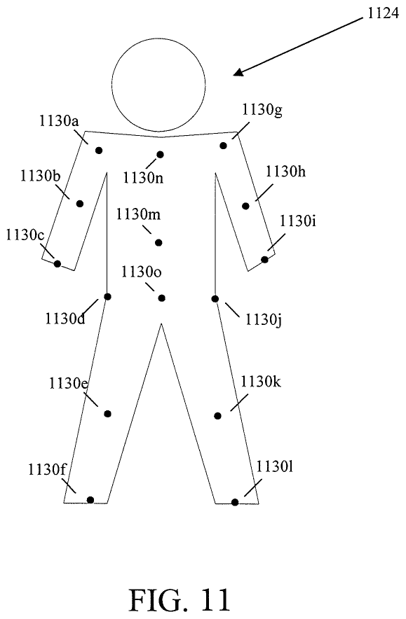

In certain embodiments, I/O devices may be formed within or otherwise associated with user's 1124 clothing or accessories, including a watch, armband, wristband, necklace, shirt, shoe, or the like. These devices may be configured to monitor athletic movements of a user. It is to be understood that they may detect athletic movement during user's 1124 interactions with computer device 1114 and/or operate independently of computer device 1114 (or any other device disclosed herein). For example, one or more devices in BAN 1102 may be configured to function as an all-day activity monitor that measures activity regardless of the user's proximity or interactions with computer device 1114. It is to be further understood that the sensory system 1302 shown in FIG. 9 and the device assembly 1400 shown in FIG. 10, each of which are described in the following paragraphs, are merely illustrative examples.

i. Shoe-Mounted Device

In certain embodiments, device 126 shown in FIG. 7, may comprise footwear which may include one or more sensors, including but not limited to those disclosed herein and/or known in the art. FIG. 9 illustrates one example embodiment of a sensor system 1302 providing one or more sensor assemblies 1304. Assembly 1304 may comprise one or more sensors, such as for example, an accelerometer, gyroscope, location-determining components, force sensors and/or or any other sensor disclosed herein or known in the art. In the illustrated embodiment, assembly 1304 incorporates a plurality of sensors, which may include force-sensitive resistor (FSR) sensors 1306; however, other sensor(s) may be utilized. Port 1308 may be positioned within a sole structure 1309 of a shoe, and is generally configured for communication with one or more electronic devices. Port 1308 may optionally be provided to be in communication with an electronic module 1310, and the sole structure 1309 may optionally include a housing 1311 or other structure to receive the module 1310. The sensor system 1302 may also include a plurality of leads 1312 connecting the FSR sensors 1306 to the port 1308, to enable communication with the module 1310 and/or another electronic device through the port 1308. Module 1310 may be contained within a well or cavity in a sole structure of a shoe, and the housing 1311 may be positioned within the well or cavity. In one embodiment, at least one gyroscope and at least one accelerometer are provided within a single housing, such as module 1310 and/or housing 1311. In at least a further embodiment, one or more sensors are provided that, when operational, are configured to provide directional information and angular rate data. The port 1308 and the module 310 include complementary interfaces 1314, 1316 for connection and communication.

In certain embodiments, at least one force-sensitive resistor 306 shown in FIG. 9 may contain first and second electrodes or electrical contacts 1318, 1320 and a force-sensitive resistive material 1322 disposed between the electrodes 1318, 1320 to electrically connect the electrodes 1318, 1320 together. When pressure is applied to the force-sensitive material 1322, the resistivity and/or conductivity of the force-sensitive material 1322 changes, which changes the electrical potential between the electrodes 1318, 1320. The change in resistance can be detected by the sensor system 1302 to detect the force applied on the sensor 1316. The force-sensitive resistive material 1322 may change its resistance under pressure in a variety of ways. For example, the force-sensitive material 1322 may have an internal resistance that decreases when the material is compressed. Further embodiments may utilize “volume-based resistance”, which may be implemented through “smart materials.” As another example, the material 1322 may change the resistance by changing the degree of surface-to-surface contact, such as between two pieces of the force sensitive material 1322 or between the force sensitive material 1322 and one or both electrodes 1318, 1320. In some circumstances, this type of force-sensitive resistive behavior may be described as “contact-based resistance.”

ii. Wrist-Worn Device

As shown in FIG. 10, device 1400 (which may resemble or comprise sensory device 1128 shown in FIG. 7), may be configured to be worn by user 1124, such as around a wrist, arm, ankle, neck or the like. Device 1400 may include an input mechanism, such as a depressible input button 1402 configured to be used during operation of the device 1400. The input button 1402 may be operably connected to a controller 1404 and/or any other electronic components, such as one or more of the elements discussed in relation to computer device 1114 shown in FIG. 7. Controller 1404 may be embedded or otherwise part of housing 1406. Housing 1406 may be formed of one or more materials, including elastomeric components and comprise one or more displays, such as display 1408. The display may be considered an illuminable portion of the device 1400. The display 1408 may include a series of individual lighting elements or light members such as LED lights 1410. The lights may be formed in an array and operably connected to the controller 1404. Device 1400 may include an indicator system 1412, which may also be considered a portion or component of the overall display 1408. Indicator system 1412 can operate and illuminate in conjunction with the display 1408 (which may have pixel member 1414) or completely separate from the display 1408. The indicator system 1412 may also include a plurality of additional lighting elements or light members, which may also take the form of LED lights in an exemplary embodiment. In certain embodiments, indicator system may provide a visual indication of goals, such as by illuminating a portion of lighting members of indicator system 1412 to represent accomplishment towards one or more goals. Device 1400 may be configured to display data expressed in terms of activity points or currency earned by the user based on the activity of the user, either through display 1408 and/or indicator system 1412.

A fastening mechanism 1416 can be disengaged wherein the device 1400 can be positioned around a wrist or portion of the user 1124 and the fastening mechanism 1416 can be subsequently placed in an engaged position. In one embodiment, fastening mechanism 1416 may comprise an interface, including but not limited to a USB port, for operative interaction with computer device 1114 and/or devices, such as devices 1120 and/or 1112. In certain embodiments, fastening member may comprise one or more magnets. In one embodiment, fastening member may be devoid of moving parts and rely entirely on magnetic forces.

In certain embodiments, device 1400 may comprise a sensor assembly (not shown in FIG. 10). The sensor assembly may comprise a plurality of different sensors, including those disclosed herein and/or known in the art. In an example embodiment, the sensor assembly may comprise or permit operative connection to any sensor disclosed herein or known in the art. Device 1400 and or its sensor assembly may be configured to receive data obtained from one or more external sensors.

iii. Apparel and/or Body Location Sensing

Element 1130 of FIG. 7 shows an example sensory location which may be associated with a physical apparatus, such as a sensor, data acquisition unit, or other device. Yet in other embodiments, it may be a specific location of a body portion or region that is monitored, such as via an image capturing device (e.g., image capturing device 1118). In certain embodiments, element 1130 may comprise a sensor, such that elements 1130a and 1130b may be sensors integrated into apparel, such as athletic clothing. Such sensors may be placed at any desired location of the body of user 1124. Sensors 1130a/b may communicate (e.g., wirelessly) with one or more devices (including other sensors) of BAN 1102, LAN 1104, and/or WAN 1106. In certain embodiments, passive sensing surfaces may reflect waveforms, such as infrared light, emitted by image-capturing device 1118 and/or sensor 1120. In one embodiment, passive sensors located on user's 1124 apparel may comprise generally spherical structures made of glass or other transparent or translucent surfaces which may reflect waveforms. Different classes of apparel may be utilized in which a given class of apparel has specific sensors configured to be located proximate to a specific portion of the user's 1124 body when properly worn. For example, golf apparel may include one or more sensors positioned on the apparel in a first configuration and yet soccer apparel may include one or more sensors positioned on apparel in a second configuration.

FIG. 11 shows illustrative locations for sensory input (see, e.g., sensory locations 1130a-1130o). In this regard, sensors may be physical sensors located on/in a user's clothing, yet in other embodiments, sensor locations 1130a-1130o may be based upon identification of relationships between two moving body parts. For example, sensor location 1130a may be determined by identifying motions of user 1124 with an image-capturing device, such as image-capturing device 1118. Thus, in certain embodiments, a sensor may not physically be located at a specific location (such as one or more of sensor locations 1130a-1130o), but is configured to sense properties of that location, such as with image-capturing device 1118 or other sensor data gathered from other locations. In this regard, the overall shape or portion of a user's body may permit identification of certain body parts. Regardless of whether an image-capturing device is utilized and/or a physical sensor located on the user 1124, and/or using data from other devices, (such as sensory system 1302), device assembly 1400 and/or any other device or sensor disclosed herein or known in the art is utilized, the sensors may sense a current location of a body part and/or track movement of the body part. In one embodiment, sensory data relating to location 1130m may be utilized in a determination of the user's center of gravity (a.k.a, center of mass). For example, relationships between location 1130a and location(s) 1130f/1130l with respect to one or more of location(s) 1130m-1130o may be utilized to determine if a user's center of gravity has been elevated along the vertical axis (such as during a jump) or if a user is attempting to “fake” a jump by bending and flexing their knees. In one embodiment, sensor location 11306n may be located at about the sternum of user 1124. Likewise, sensor location 1130o may be located approximate to the naval of user 1124. In certain embodiments, data from sensor locations 1130m-1130o may be utilized (alone or in combination with other data) to determine the center of gravity for user 1124. In further embodiments, relationships between multiple sensor locations, such as sensors 1130m-1130o, may be utilized in determining orientation of the user 1124 and/or rotational forces, such as twisting of user's 1124 torso. Further, one or more locations, such as location(s), may be utilized as (or approximate) a center of moment location. For example, in one embodiment, one or more of location(s) 1130m-1130o may serve as a point for a center of moment location of user 1124. In another embodiment, one or more locations may serve as a center of moment of specific body parts or regions.

1. An apparatus comprising:
a computing device comprising one or more processors; and
memory storing instructions that, when executed by the one or more processors, cause the device to:
receive athletic performance data corresponding to a first workout performance by a user;
generate, based at least in part on the received athletic performance data, a first virtual shadow representing the first workout performance by the user;
store the generated first virtual shadow;
receive, from a first set of sensors associated with the device, athletic performance data corresponding to a current workout routine by the user, wherein the first set of sensors is placed at one or more locations on the user;
send, to an external eyewear device, the stored generated first virtual shadow and the athletic performance data corresponding to a current workout routine by the user;
generate, by the external eyewear device, a first user avatar based on the athletic performance data corresponding to a current workout routine by the user; and,
display, by the external eyewear device, the first virtual shadow and first user avatar.

2. The apparatus of claim 1, wherein the instructions, when executed by the one or more processors, cause the apparatus to:
comparing the first virtual shadow with the first user avatar;
generating, by the computing device, results of the compared first virtual shadow with the first user avatar; and
sending for display, to the eyewear device, the generated results of the compared first virtual shadow and first user avatar.

3. The apparatus of claim 2, wherein the generated results of the compared first virtual shadow and first user avatar represent an indication of a level of progress towards a goal of the user.

4. The apparatus of claim 1, wherein the first set of sensors are placed on at least one of the user's hands and at least one of the user's feet.

5. The apparatus of claim 1, wherein the instructions, when executed by the one or more processors, cause the apparatus to:
modify, by the computing device and based on the received athletic performance data corresponding to the current workout routine, an appearance of the first user avatar.

6. The apparatus of claim 1, wherein the instructions, when executed by the one or more processors, cause the apparatus to:
receive athletic performance data corresponding to a second workout performance by a user;
generate, based at least in part on the received athletic performance data corresponding to a second workout performance, a second virtual shadow representing the first workout performance by the user;
store the generated second virtual shadow;
send, to an external eyewear device, the stored generated second virtual shadow; and,
display, by the external eyewear device, the second virtual shadow.

7. The apparatus of claim 1, wherein the computing device further comprises a sensor assembly for measuring athletic performance data corresponding to the performance of the first workout routine.

8. The apparatus of claim 1, wherein the computing device comprises at least one of: a mobile terminal, a telephone, a music player, or a portable device.

9. The apparatus of claim 6, further comprising:
sending, by the computing device to a first lens of the external eyewear device, for display to the user, the stored first virtual shadow; and,
sending, by the computing device to a second lens of the external eyewear device, for display to the user, the stored second virtual shadow.

10. A method comprising:
collecting, by a set of one or more sensors, athletic performance data corresponding to a performance of a workout routine by a user, wherein the athletic performance data includes location data;
receiving, by a computing device, the athletic performance data and real-time information associated with the location data;
determining, based on the real-time information associated with the location data, a recommended route for the user;
sending, by the computing device to an external eyewear device, the athletic performance data and recommended route; and
displaying, by the external eyewear device, the athletic performance data and recommended route.

11. The method of claim 10, wherein the real-time information including information based on construction, traffic reports, and safety events.

12. The method of claim 10, further comprising:
displaying, by a first lens of the external eyewear device, the athletic performance data; and,
displaying, by a second lens of the external eyewear device, the recommended route.

13. The method of claim 10, wherein the athletic performance data includes at least one of: speed, distance, steps taken, or energy expenditure.

14. The method of claim 10, wherein the eyewear device comprises:
a projection display comprising a plurality of lenses; and
an indicator system comprising a plurality of lighting elements, and wherein the plurality of lighting elements are illuminated in conjunction with displaying the athletic performance data and recommended route.

15. The method of claim 10, further comprising:
displaying, by the external eyewear device, benefits of the recommended route.

16. An apparatus comprising:
one or more processors; and
memory storing instructions that, when executed by the one or more processors, cause the apparatus to:
receive athletic performance data corresponding to a first workout performance by a user;
generate, based at least in part on the received athletic performance data, a first virtual shadow representing the first workout performance by the user;
store the generated first virtual shadow;
determine, based at least in part on the received athletic performance data, a first recommended exercise program for the user;
store the first recommended exercise program;
receive, from a first sensor, athletic performance data corresponding to a current workout routine by the user;
send, to an external eyewear device, the stored generated first virtual shadow and the first recommended exercise program; and,
display, by the external eyewear device, the first virtual shadow and first recommended exercise program.

17. The apparatus of claim 16, wherein the apparatus comprises at least one of: a mobile terminal, a telephone, a music player, or a portable device.

18. The apparatus of claim 16, wherein the instructions, when executed by the one or more processors, cause the apparatus to:
generate, by the external eyewear device, a first user avatar based on the athletic performance data corresponding to a current workout routine by the user; and,
display, by the external eyewear device, the first virtual shadow, the first user avatar, and the first recommended exercise program.

19. The apparatus of claim 16, wherein the athletic performance data includes at least one of: speed, distance, steps taken, or energy expenditure.

20. The apparatus of claim 16, wherein the external eyewear device further displays the athletic performance data corresponding to a current workout routine by the user.
